(19) United States
(12) Patent Application Publication
(10) Pub. No.: US 2025/0161502 A1
(43) Pub. Date: May 22, 2025

(54) PHARMACEUTICAL COMPOSITIONS COMPRISING A 225-ACTINIUM-LABELLED COMPLEX AND A BISMUTH SEQUESTERING AGENT

(71) Applicant: Novartis AG, Basel (CH)

(72) Inventors: Elena CASTALDI, Ivrea (IT); Ilyes ZAHI, Nantes (FR); Roberto FORMENTO CAVAIER, Romano Canavese (IT); Valentina MAINERO, Ivrea (IT); Maurizio F. MARIANI, Ivrea (IT)

(21) Appl. No.: 18/836,576

(22) PCT Filed: Feb. 9, 2023
(86) PCT No.: PCT/IB2023/051159

§ 371 Date: Aug. 7, 2024

Related U.S. Application Data
(60) Provisional application No. 63/308,211, filed on Feb. 9, 2022.

(51) Int. Cl. A61K 51/04; A61K 31/198; A61K 31/395

(52) U.S. Cl. CPC A61K 51/0497; A61K 31/198; A61K 31/395

(57) ABSTRACT

The disclosure concerns a pharmaceutical composition comprising a ²²⁵Ac radiolabeled complex formed by a ²²⁵Ac radionuclide, and a target binding moiety linked to a chelating agent; and a bismuth sequestering agent, typically capable of sequestering Bi³⁺. The disclosure also concerns a method for preparing said pharmaceutical composition.

### TECHNICAL FIELD

This disclosure pertains to the field of radiopharmaceutical compounds radiolabeled with Actinium 225 (²²⁵Ac) and their radiopharmaceutical compositions.

### BACKGROUND

High-energy α-particles emitted by the decay of radioactive isotopes can be harnessed with an appropriate targeting vector to destroy malignant cells. This therapeutic strategy, known as α-therapy, is the subject of intense current research. Given the 9.9 d half-life of ²²⁵Ac, which is longer than its main daughters, and the high alpha particle emission energies from itself and the daughters, it was recognized as a potential candidate for use in cancer therapy.

Hence, there has been significant interest in the development of targeted alpha-particle therapy (TAT) for the treatment of solid tumors. The efficacy of ²²⁵Ac-PSMA-617 have been demonstrated in the treatment of prostate bone metastases. These developments have further elevated interest in the development of novel α-emission cancer treatments. Typically, TAT for solid tumors involves including an α-particle-emitting radionuclide to a tumor targeting scaffold, followed by the intravenous administration and systemic targeting of tumors and metastases. The α-particle penetration range in tissues is only a few cell diameters, ensuring that the greatest effect of tumor TAT remains within the tumor volume

²²⁵Ac decay yields six principal radionuclides progeny in the decay cascade up to stable ²⁰⁹Bi. A single ²²⁵Ac (t½=9.9 d; 5.8 MeV α particle) decay yields net 4 alpha and 3 beta⁻ decays. These daughters are ²²¹Fr (t½=4.8 m; 6.3 MeV α particle and 218 keV γ emission), ²¹⁷At (t½=32.3 ms; 7 MeV α particle), ²¹³Bi (t½=45.6 m; 6 MeV α particle, 1.4 MeV Eₘₐₓ β-particle and 440 keV γ emission), ²¹³Po (t½=3.7 μs; 8.4 MeV α particle), ²⁰⁹TI (t½=2.2 m; 1.8 MeV Eₘₐₓ β-particle and 1567 keV γ emission), ²⁰⁹Pb (t½=3.25 h; 644 keV Eₘₐₓ β-particle) and ²⁰⁹Bi (stable).

A major constraint to the clinical use of pharmaceutical composition based on ²²⁵Ac, in particular in the Radio-Ligand Therapy (RLT) field is the systemic release of the daughter nuclides which may cause radiotoxicity and/or radiochemical stability. The daughters, especially ²¹³Bi tend for example to accumulate in the kidney or in the liver as reported in J. Singh Jaggi et al. Cancer Res; 65 (11)-2005.

McDevitt et al. Applied Radiation and Isotopes 57 (841-847)-2002 state that the instability is due to the high classical recoil energy of the daughter product which breaks the molecular bonds of the chelator.

As a result, the use of pharmaceutical compositions comprising a radiopharmaceutical compound radiolabeled with ²²⁵Ac is limited because of the free daughters released. Indeed, once a too higher concentration of ²¹³Bi is reached in said pharmaceutical compositions, the latter may induce toxic issue.

Thus, there is a need to develop a pharmaceutical composition comprising a radiopharmaceutical compound radiolabelled with ²²⁵Ac in which the released ²¹³Bi is sequestered so as to limit the toxicity and render the same easily eliminated by the organism.

### SUMMARY

The applicant has surprisingly discovered that it is possible to add at least one bismuth chelator, in particular a ²¹³Bi chelator, in a ²²⁵Ac compound formulation, i.e. a pharmaceutical composition comprising ²²⁵Ac and a compound, e.g. a target binding (chemical or biological) moiety linked to a chelating agent, in order to drastically/substantially decrease the toxicity induced by the ²²⁵Ac decay.

The disclosure concerns a pharmaceutical composition comprising
(a) a ²²⁵Ac radiolabelled complex formed by
   (i) a ²²⁵Ac radionuclide, and,
   (ii) a target binding moiety linked to a chelating agent; and
(b) a bismuth sequestering agent, typically capable of sequestering Bi³⁺
(c) optionally an antioxidant, particularly chosen in the group comprising gentisic acid and salts thereof, ascorbic acid and salts thereof, and mixtures thereof.

The disclosure further concerns a pharmaceutical composition comprising
(a) a ²²⁵Ac complex formed by
   (i) a ²²⁵Ac radionuclide, and,
   (ii) a target binding moiety linked to a chelating agent; and
(b) a bismuth sequestering agent, typically capable of sequestering Bi³⁺;
(c) optionally, one or more antioxidant(s), preferably selected from the group consisting of gentisic acid and salts thereof, ascorbic acid and salts thereof, and mixtures thereof.

The disclosure also concerns a method for preparing said pharmaceutical composition(s), comprising the steps of
1) Forming a complex of the radionuclide and the chelating agent linked or the target binding organic moiety by
(1.1) providing an aqueous solution comprising the radionuclide;
(1.2) providing an aqueous solution comprising the chelating agent linked to the target binding organic moiety; and
(1.3) mixing the solutions obtained in steps (1.1) and (1.2) and heating the resulting mixture;
2) Diluting the complex solution obtained at the end of step (1) by mixing the complex solution obtained at the end of step (1) with a dilution solution,
wherein said bismuth sequestering agent is comprised in the aqueous solution of step 1.1 and/or the aqueous solution of step 1.2 and/or the dilution solution of step 2.

The disclosure further concerns a patient dose unit comprising
(a) 5-20 MBq of ²²⁵Ac, particularly 7-15 MBq,
(b) 0.15-0.80 mg of bismuth sequestering agent, particularly 0.21-0.60 mg; and
(c) optionally 9-50 mg of antioxidant(s), particularly 12.6-37.5 mg.

The disclosure further concerns a pharmaceutical composition comprising
(a) 0.5-2 MBq/mL of ²²⁵Ac, particularly 0.7-1.5 MBq/mL,
(b) 0.015-0.08 mg/mL of bismuth sequestering agent, particularly 0.02-0.06 mg/mL; and
(c) optionally 0.9-5 mg/mL of antioxidant(s), particularly 1.3-3.8 mg/mL.

### BRIEF DESCRIPTION OF THE DRAWING

FIG. 1 is an illustration of the ²²⁵Ac decay scheme.

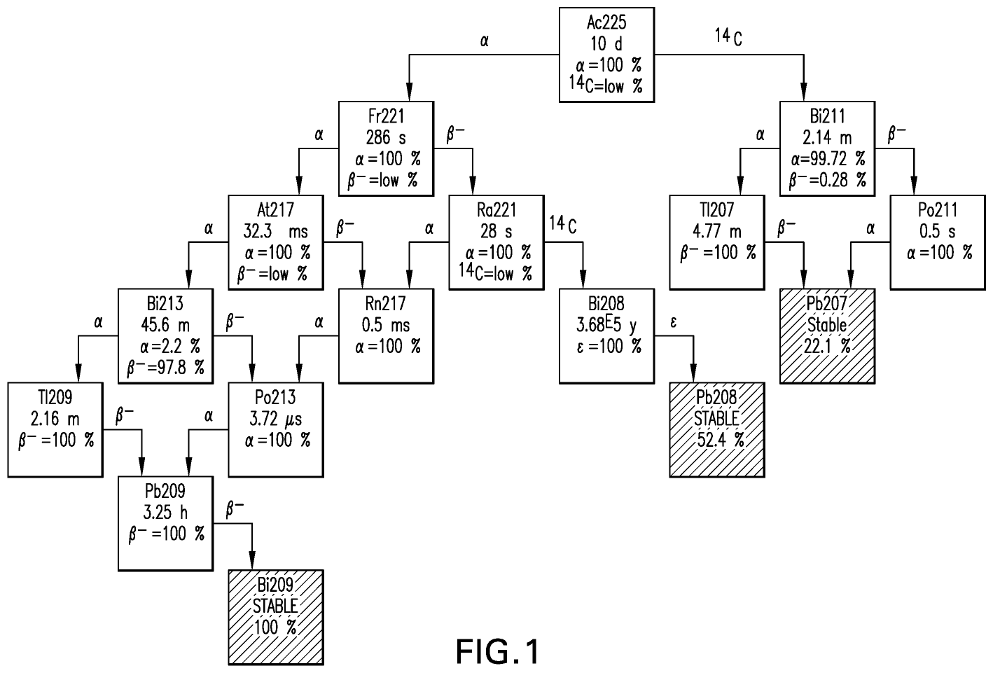

FIG. 2 is the iTLC of [²²⁵Ac]Ac-PSMA-R2 DP with DMSA.

### DETAILED DESCRIPTION

### Definitions

The use of the articles “a”, “an”, and “the” in both the description and claims are to be construed to cover both the singular and the plural, unless otherwise indicated herein or clearly contradicted by context. The terms “comprising”, “having”, “being of” as in e.g., a complex “of a radionuclide and a cell receptor binding organic moiety linked to a chelating agent”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

As used herein, the term “protecting group” refers to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting groups for protection of the carboxyl group, as described by Wutz et al. (pages 533-643), are used in certain embodiments. In some embodiments, the protecting group is removable by treatment with acid.

Representative examples of protecting groups include, but are not limited to, benzyl, p-methoxybenzyl (PMB), tertiary butyl (t-Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr). Persons skilled in the art will recognize appropriate situations in which protecting groups are required and will be able to select an appropriate protecting group for use in a particular circumstance.

As used herein, the term “aryl” refers to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphthyl and phenyl ring fused to a heterocyclyl, like benzopyranyl, benzodioxolyl, benzodioxanyl and the like.

As used herein, the terms “substituted aryl” and “substituted pyridine” refer to an aryl as defined above or a pyridine which is substituted by one or more substituents selected from: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR′″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, alkyl, heteroalkyl, cycloalkyl, heterocycloalkyl, aryl and heteroaryl. When a compound of the disclosure includes more than one R group, for example, each of the R groups is independently selected as are each R′, R″, R′″ and R″″ groups when more than one of these groups is present.

As used herein, the term “alkyl”, by itself or as part of another substituent, refers to a linear or branched alkyl functional group having 1 to 6 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the term “alkylene” refers to a divalent saturated, straight-chained or branched hydrocarbon group having 1 to 20 carbon atoms, particularly 1 to 12, more particularly 1 to 6.

As used herein, the term “heteroalkyl” refers to a linear or branched alkyl functional group having 1 to 6 carbon atoms and from 1 to 3, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule.

As used herein, the term “cycloalkyl” refers to a saturated or unsaturated cyclic group having 3 to 6 carbon atoms. Suitable cycloalkyl groups include cyclopropyl, cyclobutyl, cyclopentyl and cyclohexyl.

As used herein, the term “halogen” refers to a fluoro (—F), chloro (—Cl), bromo (—Br), or iodo (—I) group.

As used herein, the term “alkoxy” refers to a —O-alkyl group, wherein the alkyl group is a C₁-C₆ alkyl as defined herein. Suitable alkoxy groups include methoxy, ethoxy, propoxy.

As used herein, the term “heteroaryl” refers to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized, and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “heterocyclyl” or “heterocycloalkyl” refer to a saturated or unsaturated cyclic group having 5 to 10 ring atoms, wherein at least one ring atom is a heteroatom selected from N, O and S. The nitrogen and sulfur heteroatoms may optionally be oxidized and the nitrogen heteroatoms may optionally be quaternized. Examples of heterocycle include, but are not limited to, tetrahydropyridyl, piperidinyl, morpholinyl, tetrahydrofuranyl, tetrahydrothienyl, piperazinyl, 1-azepanyl, imidazolinyl, 1,4-dioxanyl and the like.

The term “about” or “ca.” has herein (unless otherwise defined in any of the paragraphs of this disclosure) the meaning that the following value may vary for ±20%, particularly ±10%, more particularly ±5%, even more particularly ±2%, even more particularly ±1%.

Unless otherwise defined, “%” has herein the meaning of weight percent (wt %), also referred to as weight by weight percent (w/w %).

Unless otherwise defined, the volumetric radioactivity expressed may vary for ±20%, particularly ±10%, particularly ±5%, even more particularly ±2%, even more particularly ±1%.

The term “total concentration” refers to the sum of one or more individual concentrations.

The term “aqueous solution” refers to a solution of one or more solute in water.

The expression “complex formed by
(i) a radionuclide, and
(ii) a target binding moiety linked to a chelating agent” corresponds to the radionuclide metal ion forming a non-covalent bond with the functional groups of the chelating agent, e.g. amines or carboxylic acids, in particular with the O-, N-, P- and/or S-atoms of functional groups. The chelating agent has at least two such complexing functional groups to be able to form a chelate complex, preferably, 3, 4, 5, 6, 7 or even 8 functional groups, more preferably, 7 or 8 functional groups.

The term “sequestering agent” refers to a chelating agent suitable to complex radionuclide metal ions and/or stable metal ions.

A “pH adjuster” is a chemical that is added to a solution to adjust a pH value of the solution and to thereby achieve a desired performance. Controlling the pH can be performed by adding a pH adjuster to the formulation. Examples of pH adjusters include commonly used acids and bases, buffers and mixtures of acids and bases. For example, bases that can be used include NaOH, KOH, Ca(OH)₂, sodium bicarbonate, potassium carbonate, and sodium carbonate. Examples of acids that can be used include hydrochloric acid, acetic acid, citric acid, formic acid, fumaric acid, and sulfamic acid. Particularly the pH adjuster can be a base, more particularly NaOH. The pH adjuster can be also TRIS, THAM, trometamol, tromethamine. The range of pH of the fluid can be any suitable range, such as about 2 to about 14.

The term “for commercial use” refers to the drug product, e.g. a pharmaceutical aqueous solution, which is able to obtain (particularly has obtained) marketing authorization by health authorities, e.g. US-FDA or EMA, by complying with all drug product quality and stability requirements as demanded by such health authorities, which is also able to be manufactured (particularly is manufactured) from or at a pharmaceutical production site at commercial scale followed by a quality control testing procedure, and is able to be supplied (particularly is supplied) to remotely located end users, e.g. hospitals or patients.

As used herein, the expression “target binding moiety” or “target binding organic moiety” refers to a part of a molecule which specifically binds with a target, typically a protein or a receptor, typically a receptor at the surface of a cell, in particular a cancerous cell.

The terms “polypeptide” and “peptide” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. In various embodiments, the polypeptide can be isolated from natural sources, can be a produced by recombinant techniques from a eukaryotic or prokaryotic host, or can be a product of synthetic procedures.

As mentioned above, the disclosure concerns a pharmaceutical composition comprising
(a) a ²²⁵Ac radiolabeled complex formed by
   (i) a ²²⁵Ac radionuclide, and,
   (ii) a target binding moiety linked to a chelating agent; and
(b) a bismuth sequestering agent, typically capable of sequestering Bi³⁺.

The disclosure further concerns a pharmaceutical composition comprising
(a) a ²²⁵Ac complex formed by
   (i) a ²²⁵Ac radionuclide, and,
   (ii) a target binding moiety linked to a chelating agent; and
(b) a bismuth sequestering agent, typically capable of sequestering Bi³⁺.

### The Radiolabeled Complex

Any radiolabeled complex, or any complex, including a ²²⁵Ac radionuclide could be a component of the pharmaceutical composition according to the disclosure. However, the pharmaceutical composition has been especially designed for a ²²⁵Ac radiolabeled complex, or a ²²⁵Ac complex, including a target binding moiety linked to a chelating agent.

Particularly, the ²²⁵Ac chelating agent can be selected from 1,4,7,10-Tetraazacyclododecane-1,4,7,10-tetraacetic acid (DOTA), 1,4,7,10-tetraazacyclododecane-1-(glutamic acid)-4,7,10-triacetic acid (DOTAGA), diethylentriamine pentaacetic acid (DTPA), nitrilotriacetic acid (NTA), ethylenediaminetetraacetic acid (EDTA), 1,4,7,10-tetraazacyclododecane-1,4,7-triacetic acid (DO3A), and 1,4,7-triazacyclononane-1,4,7-triacetic acid (NOTA), NOTAGA, particularly the chelating agent can be DOTA or DOTAGA.

The target binding moiety can be selected from the group consisting of chemical entities with a molecular weight of less than 2000 g/mol, peptides, polypeptide, proteins such as antibodies, or antigen binding fragments thereof, nanobodies, and consensus sequences from Fibronectin type III domains, peptide peptidomimetics, fusion proteins/polypeptides or low molecular weight molecules. Particularly the target binding moiety can be selected from the group consisting of PSMA binding ligands, somatostatin receptor binding peptides, gastrin-releasing peptide receptor antagonists, integrins binding ligands and fibroblast activation protein inhibitors, more particularly PSMA binding ligands. In certain embodiments of the present disclosure the target binding moiety is not an antibody. In certain embodiments of the present disclosure the target binding moiety is a chemical entities with a molecular weight of less than 2000 g/mol or a peptide.

In general, the present disclosure also concerns a pharmaceutical composition, in particular a radiopharmaceutical composition. The pharmaceutical composition is for intravenous (IV) use/application/administration. The solution is stable, concentrated, and ready-to-use.

### The Target Binding Moiety Linked to a Chelating Agent

As mentioned above, the target binding moiety can be selected among PSMA binding ligands, somatostatin receptor binding peptides, gastrin-releasing peptide receptor antagonists, integrins binding ligands and fibroblast activation protein inhibitors, particularly PSMA binding ligands.

### PSMA Binding Ligands

Particularly, the PSMA binding ligand linked to a chelating agent can be a molecule comprising a) a urea of 2 amino-acid residues, typically a glutamate-urea-lysine (GUL) moiety or a glutamate-urea-glutamate (GUG) moiety, and b) a chelating agent which can coordinate radioactive isotope, preferably said chelating agent comprises the following unit:

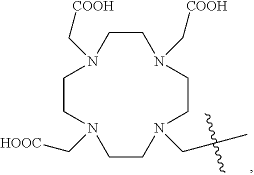 [SMILES: *CN1CCN(CC(=O)O)CCN(CC(=O)O)CCN(CC(=O)O)CC1]

wherein the chelator may be connected via a linker with the urea unit GUL or GUG and said linker may comprise residues selected from the group of Phe, Tyr, I-Tyr, 1 NaI, 2NaI, Amc, and cyclohexyl/cyclohexylene, each in unsubstituted or substituted form.

According to an embodiment, the PSMA binding ligand is a compound of formula (I):

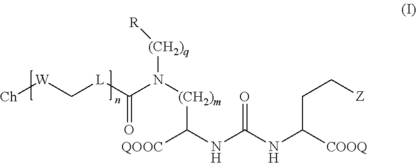

wherein:
Z is tetrazole or COOQ, particularly Z is COOQ;
Q is independently H or a protecting group, particularly Q is H;
m is an integer selected from the group consisting of 1, 2, 3, 4, and 5, particularly m is 4;
q is an integer selected from the group consisting of 1, 2, 3, 4, 5, and 6, particularly q is 1;
R is selected from the group consisting of C₆-C₁₀ aryl and heteroaryl containing 5 to 10 ring atoms, said aryl and heteroaryl being substituted 1 or more times with X;
X is —Z¹—Y;
Z¹ is a bond or a C₁-C₆ alkylene, particularly Z¹ is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂. R′, R″, R′″ and R″″ each may independently refer to hydrogen, alkyl, heteroalkyl, cycloalkyl, heterocycloalkyl, aryl and heteroaryl;
W is selected from the group consisting of —NR²—(C═O), —NR²—(C═S), —(C═O)—NR²—, and —(C═S)—NR²—, particularly, W is —(C═O)—NR²—;
each occurrence of L and W can be the same or different;
R² is H or C₁-C₄ alkyl, particularly R² is H;
n is an integer selected from the group consisting of 1, 2 and 3;
Ch is a chelating agent, particularly

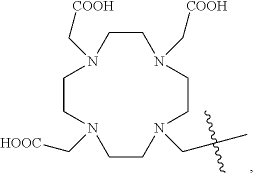 [SMILES: *CN1CCN(CC(=O)O)CCN(CC(=O)O)CCN(CC(=O)O)CC1]

Compounds of formula (I) include the stereoisomers of formulae (Ia), (Ib), (Ic) and (Id):

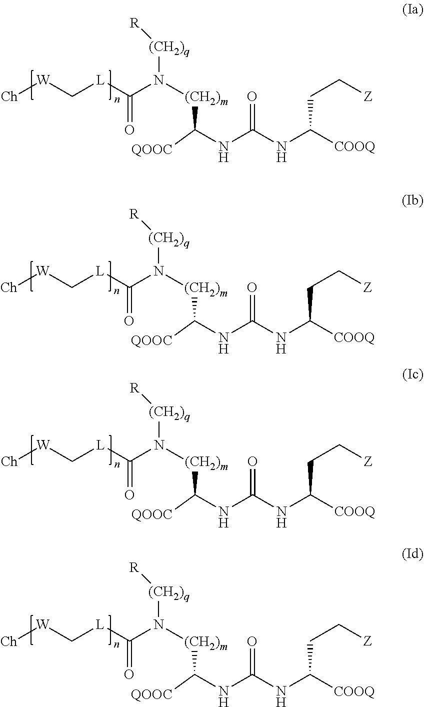

The phrase “wherein each occurrence of L and W can be the same or different” means that when the variable “n” is 2 or 3, one “L” group can be C₁-C₆ alkylene, whereas the other “L” group or groups can be C₃-C₆ cycloalkylene or arylene, or, in other embodiments, each “L” group can be, for example, C₁-C₆ alkylene. Likewise, for example, when “n” is 2 or 3, one “W” group can be —(C═O)—NR²— and the other “W” group or groups can be —(C═S)—NR²—, or, in other embodiments, each “W” can be, for example, —(C═O)—NR²—.

According to an embodiment, L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, —NR′R″, -halogen, —OC(O)R′, —C(O)R′, —CO₂R′, —C(O)NR′R″, —OC(O)NR′R″, —NR″C(O)R′, —NR′—C(O)NR″R′″, —NR″C(O)OR′. R′, R″ and R′″ each may independently refer to hydrogen, alkyl, heteroalkyl, cycloalkyl, heterocycloalkyl, aryl and heteroaryl.

According to an embodiment, L is a linker selected from the group consisting of C₃-C₆ alkylene optionally substituted with one or more substituents selected from: —OR′, ═O, ═NR′, —NR′R″, -halogen, —OC(O)R′, —C(O)R′, —CO₂R′, —C(O)NR′R″, —OC(O)NR′R″, —NR″C(O)R′, —NR′—C(O)NR″R′″, —NR″C(O)OR′. R′, R″ and R′″ each may independently refer to hydrogen, alkyl, heteroalkyl, cycloalkyl, heterocycloalkyl, aryl and heteroaryl.

According to an embodiment, R is selected from the group consisting of C₆-C₁₀ aryl substituted with one or more halogen and pyridine substituted with one or more halogen.

According to an embodiment, R is selected from the group consisting of:

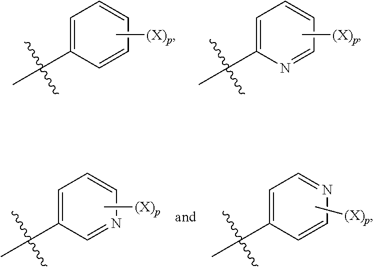

wherein p is an integer selected from the group consisting of 1, 2, 3, 4, and 5, particularly p is 1.

According to a specific embodiment, R is selected from

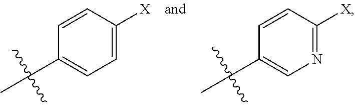

and, more particularly R can be

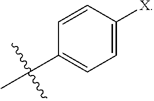

According to a specific embodiment, X is selected from Br and I.

Particularly, R is

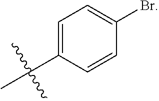 [SMILES: *c1ccc(Br)cc1]

Ch can be selected from the group consisting of:

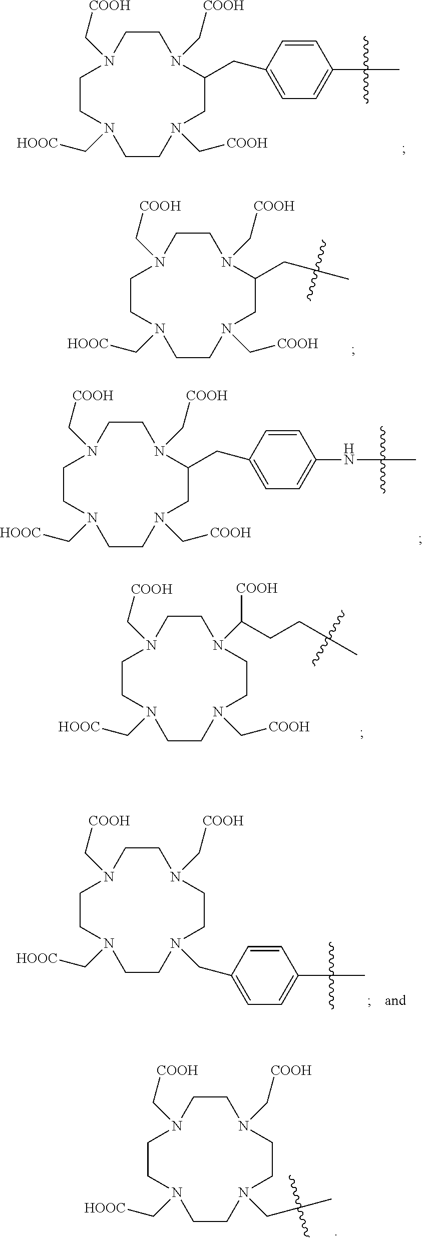

According to a specific embodiment, Ch is

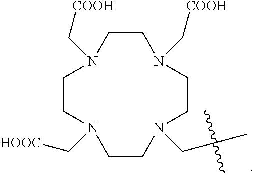 [SMILES: *CN1CCN(CC(=O)O)CCN(CC(=O)O)CCN(CC(=O)O)CC1]

According to an embodiment, W is —(C═O)—NR²—, and Ch is

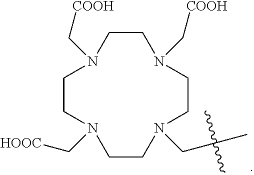 [SMILES: *CN1CCN(CC(=O)O)CCN(CC(=O)O)CCN(CC(=O)O)CC1]

According to an embodiment, m is 4, Z is COOQ, and Q is H.

In specific embodiments, R is

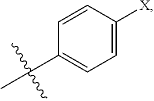

### and Ch is

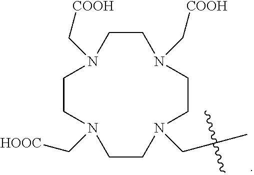 [SMILES: *CN1CCN(CC(=O)O)CCN(CC(=O)O)CCN(CC(=O)O)CC1]

According to a particular embodiment, the PSMA binding ligand is a compound of formula (II):

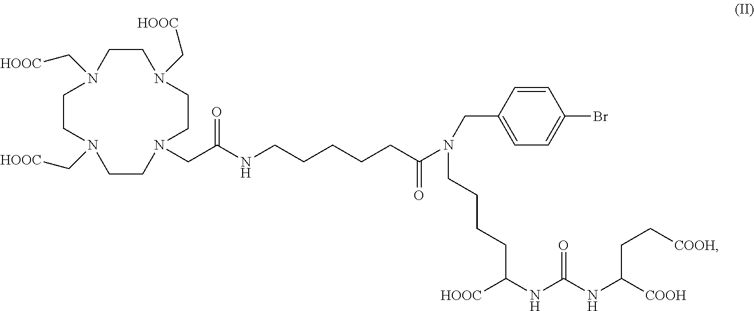 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

preferably with the Glu residue and the Lys residue being in the L-configuration.

According to another embodiment, the PSMA binding ligand is a compound of formula (III):

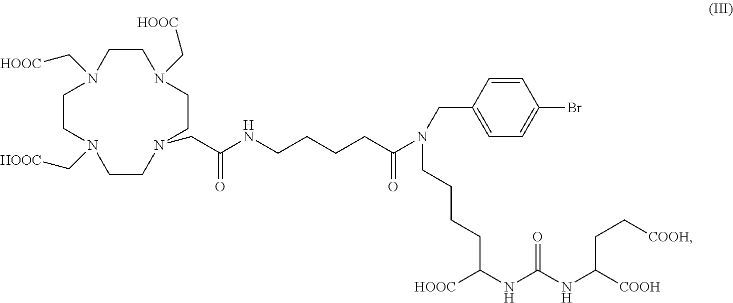 [SMILES: O=C(O)CCC(NC(=O)NC(CCCCN(Cc1ccc(Br)cc1)C(=O)CCCCNC(=O)CN1CCN(CC(=O)O)CCN(CC(=O)O)CCN(CC(=O)O)CC1)C(=O)O)C(=O)O]

preferably with the Glu residue and the Lys residue being in the L-configuration.

According to another embodiment, the PSMA-binding ligand include PSMA-617 of formula (IV) below and PSMA I&T of formula (IV′) below:

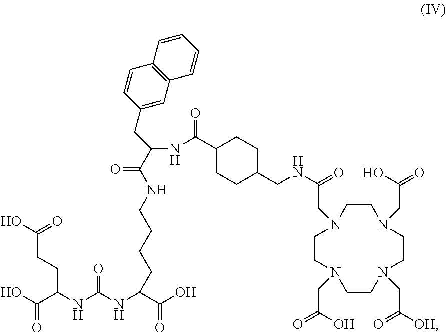 [SMILES: O=C(O)CCC(NC(=O)NC(CCCCNC(=O)C(Cc1ccc2ccccc2c1)NC(=O)C1CCC(CNC(=O)CN2CCN(CC(=O)O)CCN(CC(=O)O)CCN(CC(=O)O)CC2)CC1)C(=O)O)C(=O)O]

preferably with the Glu residue, the Lys residue, and the 2-Nal residue being in the L-configuration, and the cyclohexyl unit preferably in trans-conformation,

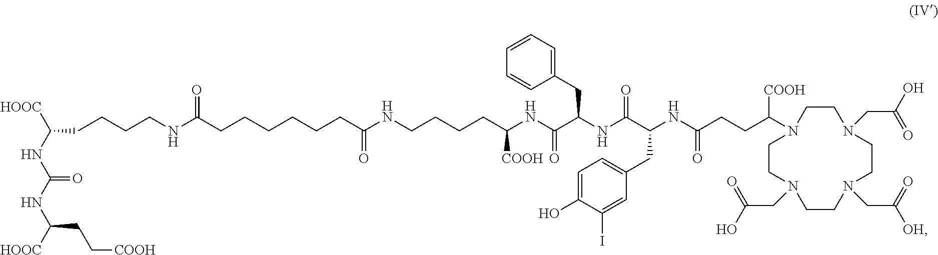 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

preferably with the glutamic acid residue next to the DOTA unit in the L-configuration.

According to another embodiment, the PSMA binding ligand is selected from the group consisting of PSMA-617 (vipivotide tetraxetan), PSMA I&T (zadavotide guraxetan, DOTAGA-(l-y)fk(Sub-KuE)), PSMA-R2, MIP-1095, MIP-1545, MIP-1555, MIP-1557, MIP-1558, CTT1403, FC705, BAY-2315497, TLX592, PSMA-TCC, rhPSMA, rhPSMA-7, rhPSMA-7.3, rhPSMA-10.1, Ludotadipep, PNT2001, PNT2002, PSMA-7 I&T, EB-PSMA-617, PSMA-ALB-02, PSMA-ALB-053, PSMA-ALB-056, P16-093, PSMA-93, and RPS-074, or an antibody or fragment thereof, e.g. TLX591, J591, rosopatamab, IAB2M, GCP-05, 1H8H5, SP29, or FOLHI, preferably selected from the group consisting of PSMA-617, PSMA I&T, and PSMA-R2.

### Synthesis of the Compounds of Formula (I), (II) and (III)

The compounds of formula (I), (II) and (III) can be synthesized using the methods disclosed in WO2017/165473.

In particular, the compound of formula (II) can be synthesized as disclosed in scheme 1. The p-bromobenzyl group modified of Glu-Lys urea 2 can be prepared by reductive alkylation of Glu-Lys urea 1 with p-bromobenzaldehyde in presence of sodium cyanoborohydride in methanol. This procedure has been described in the literature (Tykvart et al. (2015) Journal of medicinal chemistry 58, 4357-63). Then, an aliphatic linker, Boc-6-aminohexanoic acid can be coupled on the same ε-Lys amine of 2, for example using a base (like N,N-diisopropylethylamine) and a coupling agent (like N,N,N′,N′-Tetramethyl-O—(N-succinimidyl)uronium tetrafluoroborate or 1-[Bis(dimethylamino)methylene]-1H-1,2,3-triazolo[4,5-b]pyridinium 3-oxid hexafluorophosphate), to yield compound 3. Compound 3 can then be deprotected to yield compound 4, for example using an acid like trifluoroacetic acid. Finally, conjugation with commercially available DOTA-NHS ester can be performed to yield compound (II).

### Scheme 1: Synthesis of the Compound of Formula (II)

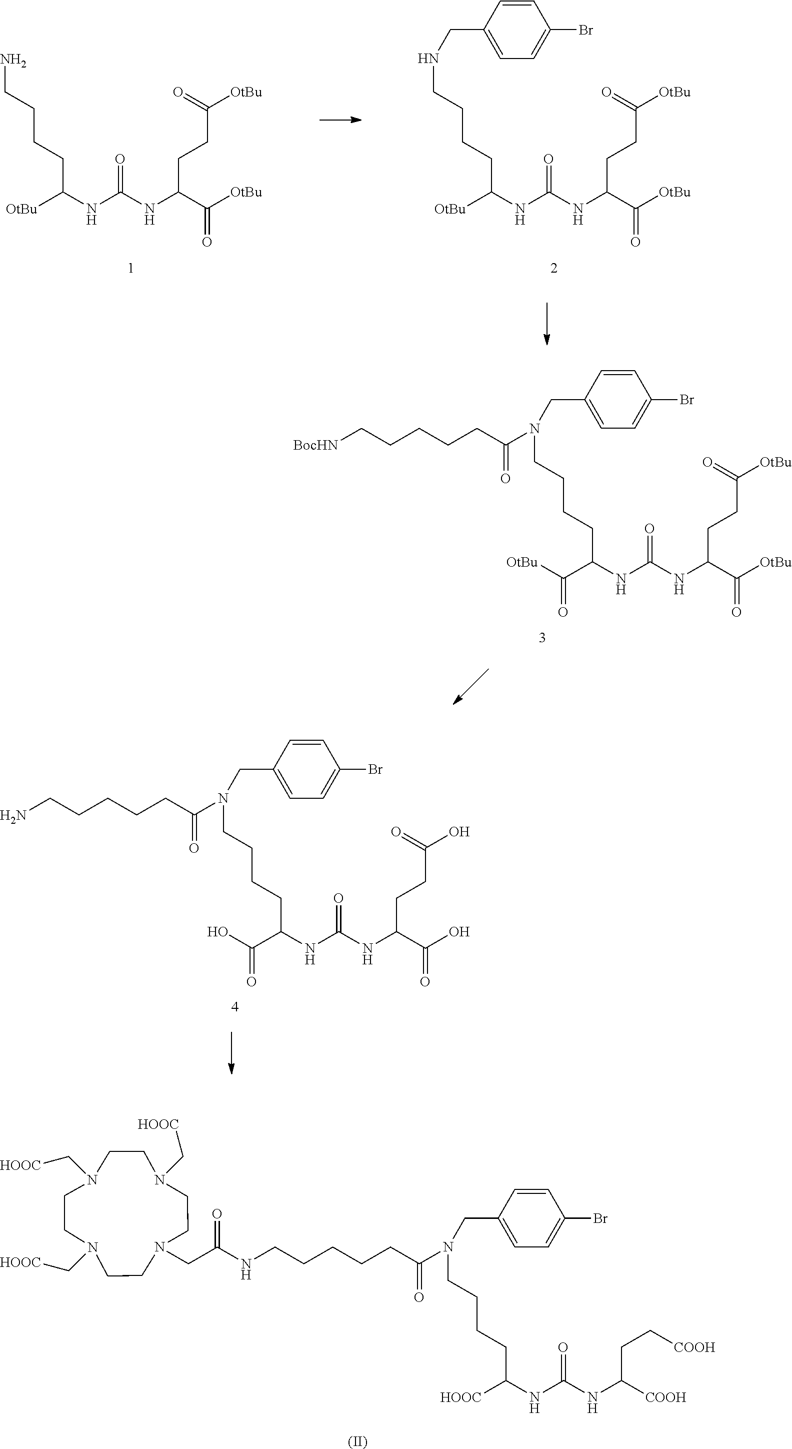 [SMILES: CC(C)(C)OC(=O)CCC(NC(=O)NC(CCCCN)OC(C)(C)C)C(=O)OC(C)(C)C ; CC(C)(C)OC(=O)CCC(NC(=O)NC(CCCCNCc1ccc(Br)cc1)OC(C)(C)C)C(=O)OC(C)(C)C ; CC(C)(C)OC(=O)CCC(NC(=O)NC(CCCCN(Cc1ccc(Br)cc1)C(=O)CCCCCNC(=O)OC(C)(C)C)C(=O)OC(C)(C)C)C(=O)OC(C)(C)C ; NCCCCCC(=O)N(CCCCC(NC(=O)NC(CCC(=O)O)C(=O)O)C(=O)O)Cc1ccc(Br)cc1 ; O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O ; "(II)"]

### The Somatostatin Receptor Binding Peptides

A somatostatin receptor (SSTR) binding is a compound which has specific binding affinity to somatostatin receptor. As used herein, the term “somatostatin receptor binding peptide” refers to a peptidic moiety with specific binding affinity to somatostatin receptor.

Particularly, said somatostatin receptor binding peptide can be a compound of formula C—S—P wherein:
C is a chelating agent;
S is an optional spacer covalently linked between C and P;
P is a somatostatin receptor binding peptide covalently linked to C, for example via its N-terminal end, either directly or indirectly via S.

Said somatostatin receptor binding peptide may be selected from octreotide, edotreotide, oxodotreotide, octreotate, lanreotide, vapreotide, satoreotide, and pasireotide.

The chelating agent C is either directly linked to the somatostatin receptor binding peptide or connected via a linker molecule, particularly it is directly linked. The linking bond(s) is (are) either covalent or non-covalent bond(s) between the cell receptor binding organic moiety (and the linker) and the chelating agent, particularly the bond(s) is (are) covalent. The chelating agent C in the context of the present disclosure is particularly selected in the group comprising 1,4,7,10-Tetraazacyclododecane-1,4,7,10-tetraacetic acid (DOTA), diethylentriaminepentaacetic acid (DTPA), nitrilotriacetic acid (NTA), ethylenediaminetetraacetic acid (EDTA), 1,4,7,10-tetraazacyclododecane-1,4,7-triacetic acid (DO3A), triethylenetetramine TETA, 1,4,7-triazacyclononane-1,4,7-triacetic acid (NOTA). In many embodiments of the disclosure, the chelating agent is DOTA.

According to many embodiments of present disclosure, the somatostatin receptor binding peptide linked to the chelating agent is selected from DOTA-OC, DOTA-TOC, DOTA-NOC, DOTA-TATE, DOTA-LAN, and DOTA-VAP. In many of these embodiments, the somatostatin receptor binding peptide is DOTA-TOC (edotreotide) or DOTA-TATE (oxodotreotide) or satoreotide tetraxetan or satoreotide trizoxetan. In many such embodiments, the somatostatin receptor binding peptide can be DOTA-TATE.

### The Gastrin-Releasing Peptide Receptor Antagonists

Particularly, said gastrin-releasing peptide receptor antagonist (GRPR antagonist) linked to a chelating agent can have the following formula Ch′-S′-P′ wherein:
Ch′ is a chelator,
S′ is an optional spacer covalently linked between C and the N-terminal of P;
P′ is a GRP receptor peptide antagonist, particularly of the general formula:

Xaa1-Xaa2-Xaa3-Xaa4-Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or p-Ala;
Xaa7 is His or (3-methyl)histidine (3-Me)His;
Z is selected from —NHOH, —NHNH2, —NH-alkyl, —N(alkyl)2, and —O-alkyl or Z is

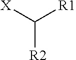

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group.

According to an embodiment, Z is selected from one of the following formulae, wherein X is NH or O:

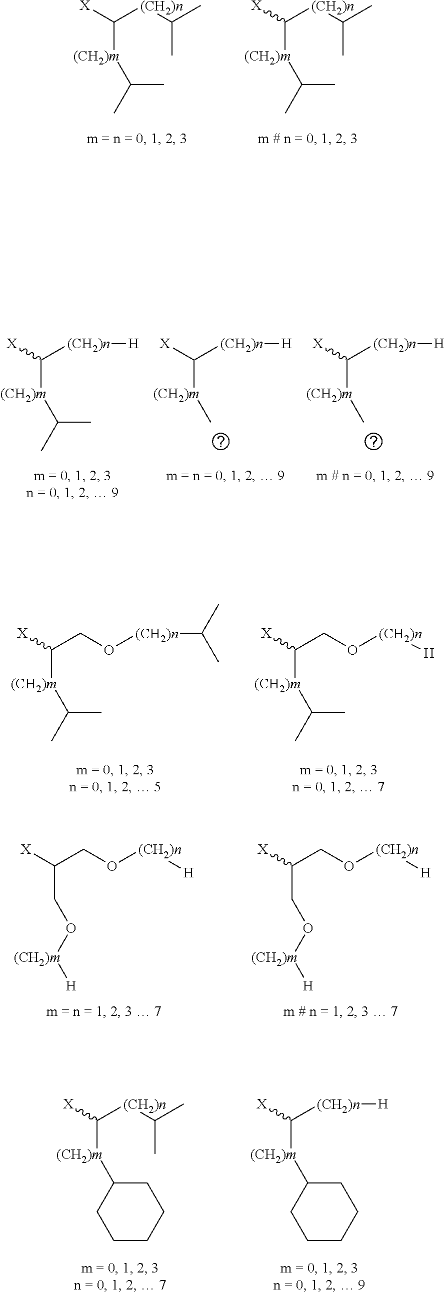

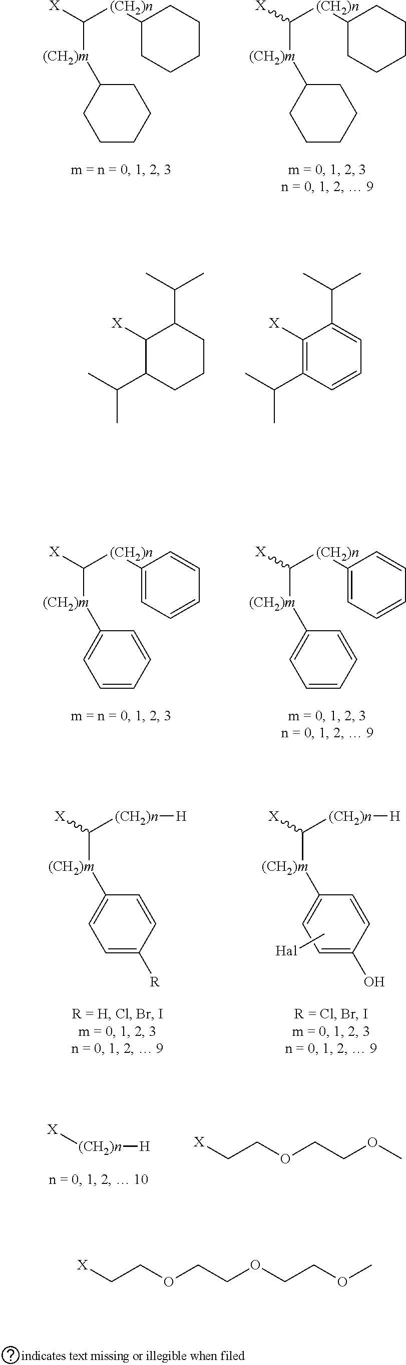

According to an embodiment, P′ is DPhe-Gln-Trp-Ala-Val-Gly-His-Z; wherein Z is defined as above.

According to an embodiment, P′ is DPhe-Gln-Trp-Ala-Val-Gly-His-Z; Z is selected from Leu-ψ(CH2N)-Pro-NH2 and NH—CH(CH₂—CH(CH₃)₂)₂ or Z is

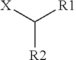

wherein X is NH (amide) and R2 is (CH₂—CH(CH₃)₂ and R1 is the same as R2 or different (CH2N)-Pro-NH2.

According to an embodiment, the chelator Ch′ is obtained by grafting one chelating agent selected among the following list:

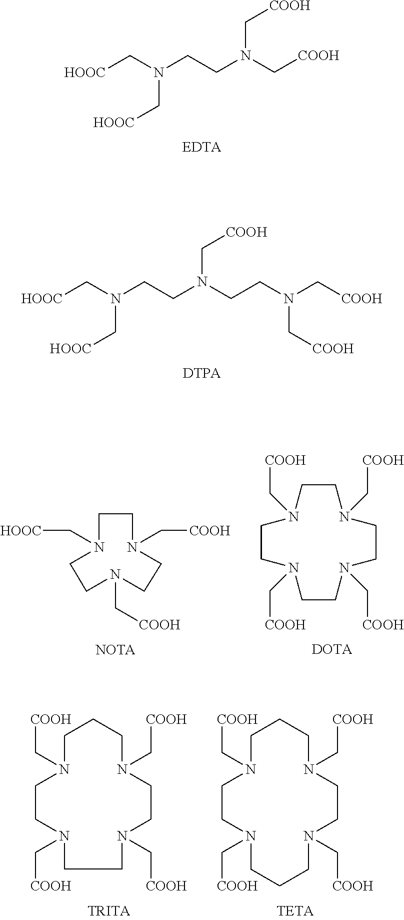

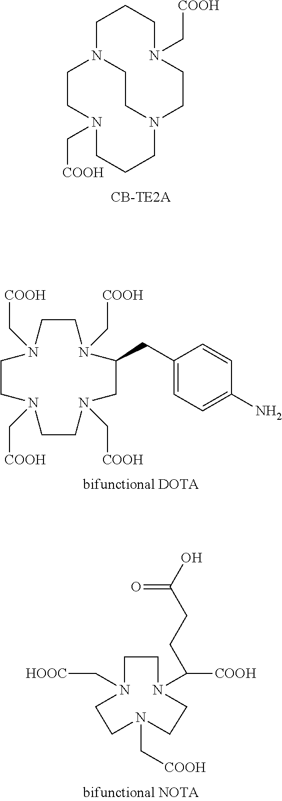 [SMILES: O=C(O)CN1CCCN2CCN(CCCN(CC(=O)O)CC2)CC1 ; Nc1ccc(C[C@H]2CN(CC(=O)O)CCN(CC(=O)O)CCN(CC(=O)O)CCN2CC(=O)O)cc1 ; O=C(O)CCC(C(=O)O)N1CCN(CC(=O)O)CCN(CC(=O)O)CC1]

According to an embodiment, the chelator Ch′ is selected from the group consisting of DOTA, DTPA, NTA, EDTA, DO3A, and NOTA, particularly is DOTA.

According to an embodiment, 8′ is selected from the group consisting of:
a) aryl containing residues of the formulae:

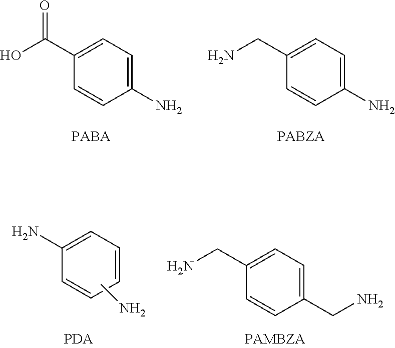

wherein PABA is p-aminobenzoic acid, PABZA is p-aminobenzylamine, PDA is phenylenediamine and PAMBZA is (aminomethyl) benzylamine;
b) dicarboxylic acids, ω-aminocarboxylic acids, ω-diaminocarboxylic acids or diamines of the formulae:

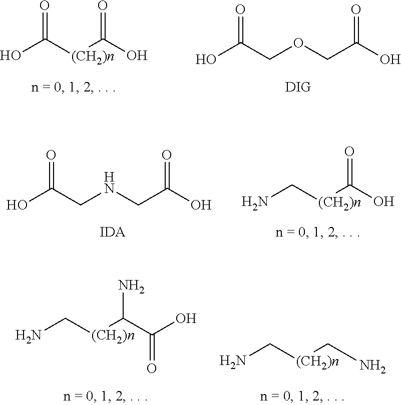

wherein DIG is diglycolic acid and IDA is iminodiacetic acid;
c) PEG spacers of various chain lengths, in particular PEG spacers selected from:

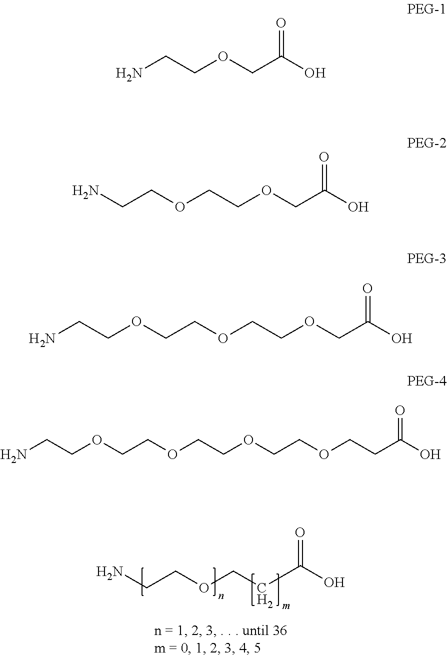

d) α- and β-amino acids, single or in homologous chains various chain lengths or heterologous chains of various chain lengths, in particular:

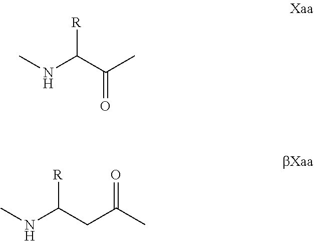

GRP(1-18), GRP(14-18), GRP(13-18), BBN(I-5), or [Tyr4]BB (1-5); or
e) combinations of a, b, c and d.

According to an embodiment, the GRPR antagonist linked to a chelating agent is selected from the group consisting of compounds of the following formulae:

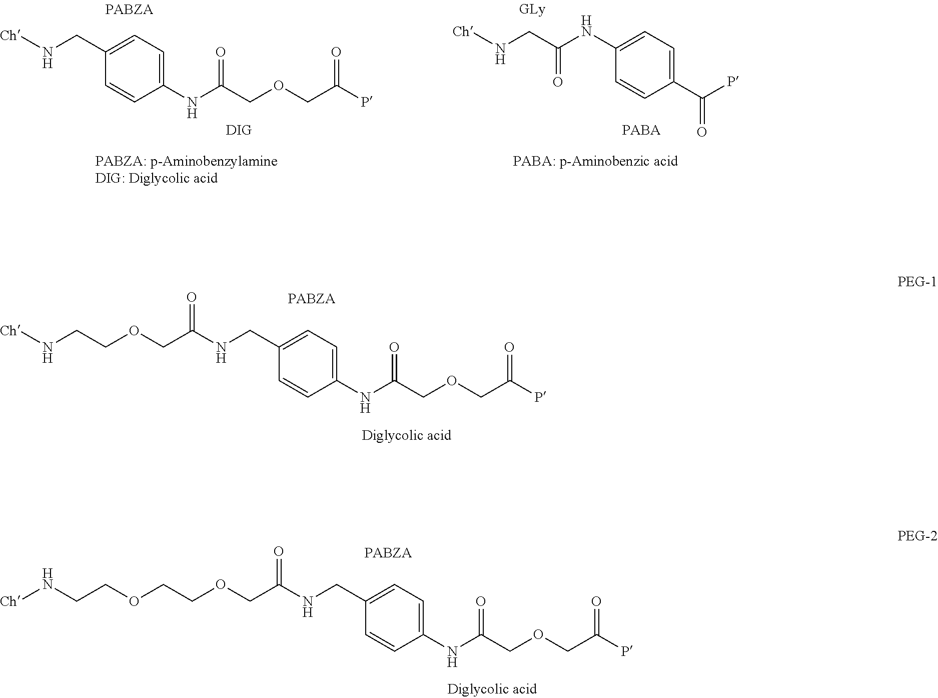

wherein Ch′ and P′ are as defined above.

According to an embodiment P′ is DPhe-Gln-Trp-Ala-Val-Gly-His-NH—CH(CH2-CH(CH3)₂)₂.

According to an embodiment, said GRPR antagonist linked to a chelating agent is NeoB1 of formula (V):

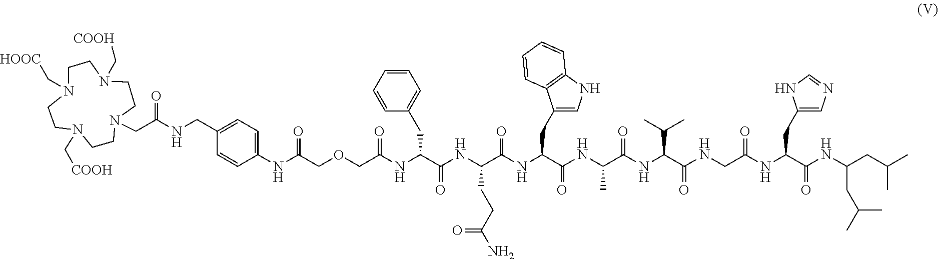 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

(DOTA-(p-aminobenzylamine-diglycolic acid)-[D-Phe-Gln-Trp-Ala-Val-Gly-His-NH—CH[CH2-CH(CH3)₂]₂;

### The Integrin Binding Ligands

Integrins are heterodimeric receptors that are important for cell-cell and cell-extracellular matrix (ECM) interactions and are composed of one α and one β-subunit.

In embodiments, the integrin binding ligand is an αvβ3 or αvβ5 integrins antagonist.

In a particular embodiment, the integrin binding ligand is of the following formula (VI):

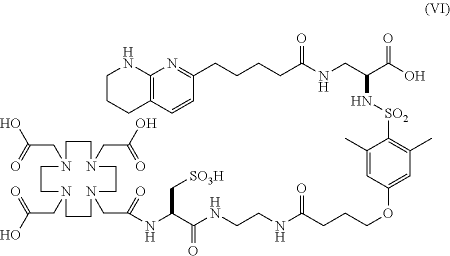

### The Fibroblast Activation Protein Inhibitors

The fibroblast activation protein inhibitor is particularly of Formula (VII):

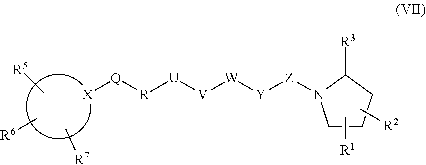

wherein
Q, R, U, V, W, Y, Z are individually present or absent under the proviso that at least three of Q, R, U, V, W, Y, Z are present;
Q, R, U, V, W, Y, Z are independently selected form the group consisting of O, CH₂, NR⁴, C=0, C═S, C═NR⁴, HCR⁴ and R⁴ CR⁴, with the proviso that two Os are not directly adjacent to each other; particularly out of the six four groups are present of which two are C=0, one is CH2 and one is NH; more particularly four groups are present of which two are C=0, one is CH2 and one is NH; most particularly, V, W, Y and Z are present of which V and Z are C=0 and W and Y are independently selected from CH2 and NH;
R¹ and R² are independently selected from the group consisting of —H, —OH, halo, C₁₋₆-alkyl, -
O—C₁₋₆-alkyl, S—C₁₋₆-alkyl;
R³ is selected from the group consisting of —H, —CN, —B(OH)₂, —C(O)-alkyl, —C(O)-aryl-, —C═C—C(O)-aryl, —C═C—S(O)₂-aryl, —CO₂H, —SO₂H, —SO₂ NH₂, —PO₃ H₂, and 5-tetrazolyl;
R⁴ is selected from the group consisting of —H, —C₁₋₆-alkyl, —O—C₁₋₆-alkyl, —S—C₁₋₆-alkyl, alkenyl, heteroalkenyl, cycloalkenyl, cycloheteroalkenyl, alkynyl, aryl, and —C₁₋₆-aralkyl, each of said —C₁₋₆-alkyl being optionally substituted with from 1 to 3 substituents selected from —OH, oxo, halo and optionally connected to Q, R, U, V, W, Y or Z;
R⁵ is selected from the group consisting of —H, halo and C₁₋₆-alkyl;
R⁶, and R⁷ are independently selected from the group consisting of —H,

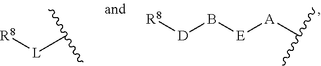

under the proviso that R⁶ and R⁷ are not at the same time H, particularly R⁶ is attached to the 7- or 8-quinolyl position and R⁷ is attached to the 5- or 6-quinolyl position; more particularly R⁶ is attached to the 7-quinolyl position and R⁷ is attached to the 6-quinolyl position,
wherein L is a linker,
wherein D, A, E, and B are individually present or absent, particularly wherein at least A, E, and B are present, wherein when present:
D is a linker;
A is selected from the group consisting of NR⁴, O, S, and CH₂;
E is selected from the group consisting of:
C₁₋₆-alkyl,

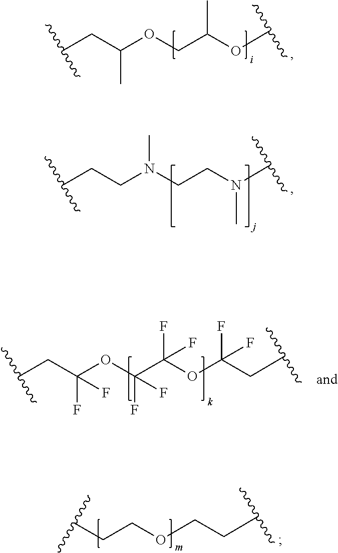

wherein i is 1, 2, or 3;
wherein j is 1, 2, or 3;
wherein k is 1, 2, or 3;
wherein m is 1, 2, or 3;
more particularly, E can be C₁₋₆-alkyl, most particularly, E is C3 or C4 alkyl;
A and E together form a group selected from: a cycloalkyl, heterocycloalkyl, aryl and heteroaryl, particularly heterocycloalkyl, wherein A and E can be mono-, bi- and multicyclic, particularly monocyclic. Each A and E being optionally substituted with 1 to 4 substituents selected from —H, —C₁₋₆-alkyl, —O—C₁₋₆-alkyl, —S—C₁₋₆-alkyl, alkenyl, heteroalkenyl, cycloalkenyl, cycloheteroalkenyl, alkynyl, aryl, and —C₁₋₆-aralkyl, each of said —C₁₋₆-alkyl being optionally substituted with from 1 to 3 substituents selected from —OH, oxo, halo; and optionally connected to A, B, D, E or

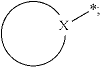

B is selected from the group consisting of S, NR⁴, NR⁴—O, NR⁴—C₁₋₆-alkyl, NR⁴—C₁₋₆-alkyl-NR⁴, and a 5- to 10-membered N-containing aromatic or non-aromatic mono- or bicyclic heterocycle, particularly further comprising 1 or 2 heteroatoms selected from O, N, and S, particularly further comprising 1 or 2 nitrogen atoms, particularly wherein NR⁴, —C₁₋₆-alkyl-NR⁴ and the N-containing heterocycle is substituted with 1 to 3 substituents selected the group consisting of C₁₋₆-alkyl, aryl, C₁₋₆-aralkyl; and

R⁸ is selected from the group consisting of radioactive moiety, chelating agent, fluorescent dye, a contrast agent and combinations thereof;

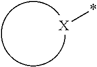

is a 1-naphtyl moiety or a 5 to 10-membered N-containing aromatic or non-aromatic mono- or bicyclic heterocycle, wherein there are 2 ring atoms between the N atom and X; said heterocycle optionally further comprising 1, 2 or 3 heteroatoms selected from O, N and S; and X is a C atom; or a pharmaceutically acceptable tautomer, racemate, hydrate, solvate, or salt thereof. Particularly, C₁₋₆-alkyl can be selected from the group consisting of methyl, ethyl, propyl, i-propyl, butyl, sec-butyl, tert-butyl, pentyl and hexyl.

In a particular embodiment, A and E together form a group selected from the group consisting of a C₃, C4, C5, C6, C7 and C8 monocyclic, particularly C5 or C6 monocyclic, or C7, C8, C9, C10, C11 or C12 bicyclic, particularly C7, C8, C9 and C10 bicyclic heterocycloalkyl, comprising 1, 2, 3, or 4, particularly 1 or 2 heteroatoms independently selected from the group consisting of N, O and S, particularly N and 0, most particularly 1 or 2 N.

In a specific embodiment, fibroblast activation protein inhibitor (FAPi) is

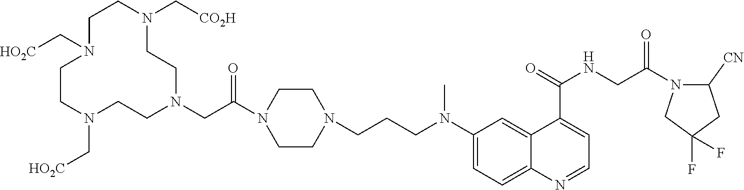 [SMILES: CN(CCCN1CCN(C(=O)CN2CCN(CC(=O)O)CCN(CC(=O)O)CCN(CC(=O)O)CC2)CC1)c1ccc2nccc(C(=O)NCC(=O)N3CC(F)(F)CC3C#N)c2c1]

In certain embodiments, the FAPi, is any one as disclosed in WO 2021/005131, WO 2021/005125, WO 2022/148851, WO 2022/148843, WO 2023/002045, the disclosure of which is incorporated herein by reference in its entirety.

In particular, the FAPi is FAP-2286/3BP-3554 (Hex-[Cys(tMeBn(DOTA-AET))-Pro-Pro-Thr-Gln-Phe-Cys]-OH)

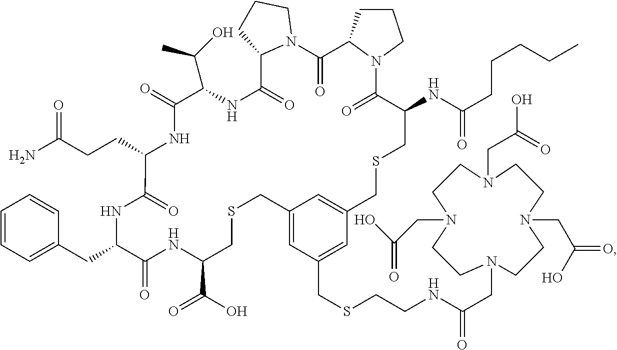 [SMILES: CCCCCC(=O)N[C@H]1CSCc2cc(CSCCNC(=O)CN3CCN(CC(=O)O)CCN(CC(=O)O)CCN(CC(=O)O)CC3)cc(c2)CSC[C@@H](C(=O)O)NC(=O)[C@H](Cc2ccccc2)NC(=O)[C@H](CCC(N)=O)NC(=O)[C@H]([C@@H](C)O)NC(=O)[C@@H]2CCCN2C(=O)[C@@H]2CCCN2C1=O]

or
3BP-3940, nBu-CAyl-[Cys(tMeBn(DOTA-AET))-Pro-Pro-Thr-Gln-Phe-Cys]-OH

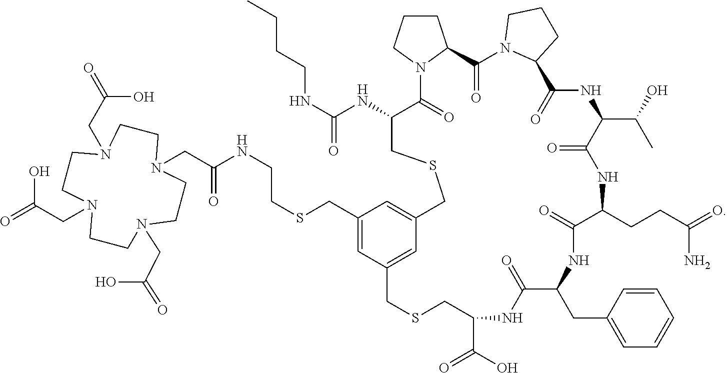 [SMILES: CCCCNC(=O)N[C@H]1CSCc2cc(CSCCNC(=O)CN3CCN(CC(=O)O)CCN(CC(=O)O)CCN(CC(=O)O)CC3)cc(c2)CSC[C@@H](C(=O)O)NC(=O)[C@H](Cc2ccccc2)NC(=O)[C@H](CCC(N)=O)NC(=O)[C@H]([C@@H](C)O)NC(=O)[C@@H]2CCCN2C(=O)[C@@H]2CCCN2C1=O]

In certain embodiments of the present disclosure, the target binding moiety linked with a chelating agent is a PSMA binding ligand and not a SSTR binding ligand, not a GRPR antagonists, not a FAPi. The present disclosure is considered in particular useful for PSMA binding ligand, in particular PSMA-617, PSMA I&T and PSMA-R2, in particular PSMA-617 and PSMA-R2, in particular PSMA-R2.

### The Bismuth Sequestering Agent

The bismuth sequestering agent could be any chelate/chelator/chelating agent capable of stably/irreversively chelating bismuth. However, while not necessary, it is advantageous that the sequestering agent selectively chelates Bismuth over Actinium.

As such, said sequestering agent is particularly a chelating agent for Bi³⁺ having binding kinetics for Bi³⁺ which is higher (preferably by a factor of 2, 3, 4, 5, 6, 7, 8, 9, 10, or even more higher) than the corresponding binding kinetics of DTPA and/or DOTA, particularly DOTA.

As already mentioned, the sequestering agent is particularly a chelating agent having binding selectivity of Bi³⁺ over Ac³⁺, particularly with a binding kinetics ratio of at least 80, particularly at least 90, for example between 90 and 100, particularly between 95 and 100, and more particularly between 98 and 100.

In one particular embodiment, the bismuth sequestering agent is a chelating agent selected from the group consisting of DSMA (also referred to as DMSA), DMPS, DOTA, DTPA, CHX-A″″-DTPA, EDTA, L^{py}, L^{pyd}, L^{pyr}, L^{pz}, NETA, 3p-C-NETA, DEPA, 3p-C-DEPA, C-DEPA, and more particularly Meso-2,3-dimercaptosuccinic acid (DMSA). In certain embodiments, DTPA is not used as bismuth sequesting agent, preferably, in certain embodiments, DTPA is not used at all in the pharmaceutical compositions.

### Other Features of the Pharmaceutical Composition According to the Disclosure

In a particular embodiment, said radionuclide is present at a concentration so that it provides a volumetric radioactivity of at least 5 MBq/mL, particularly at least 2.5 MBq/mL, and more particularly at least 1 MBq/mL (at EOP) (±10%).

Particularly, the molar ratio (i) the ²²⁵Ac radiolabelled complex to (ii) the bismuth sequestering agent can be comprised between 1:8500 and 1:80000. In particular embodiments the bismuth sequestering agent is present in the pharmaceutical composition in a concentration of from 7 to 70 μg/mL.

In certain embodiments, the pharmaceutical composition of the present disclosure comprises ²²⁵Ac in volumetric activity of from about 0.1 MBq/mL to about 10 MBq/mL and one or more bismuth sequestering agent(s) in a total concentration of from about 0.005 mg/mL to about 1 mg/mL. Preferably, in certain embodiments, the pharmaceutical composition of the present disclosure comprises ²²⁵Ac in volumetric activity of from about 0.5 MBq/mL to about 5 MBq/mL and one or more bismuth sequestering agent(s) in a total concentration of from about 0.01 mg/mL to about 5 mg/mL. More preferably, in certain embodiments, the pharmaceutical composition of the present disclosure comprises ²²⁵Ac in volumetric activity of from about 0.8 MBq/mL to about 1.5 MBq/mL and one or more bismuth sequestering agent(s) in a total concentration of from about 0.02 mg/mL to about 2 mg/mL. Even more preferably, in certain embodiments, the pharmaceutical composition of the present disclosure comprises ²²⁵Ac in volumetric activity of from about 0.9 MBq/mL to about 1.2 MBq/mL and one or more bismuth sequestering agent(s) in a total concentration of from about 0.03 mg/mL to about 1 mg/mL. Even more preferably, in certain embodiments, the pharmaceutical composition of the present disclosure comprises ²²⁵Ac in volumetric activity of from about 1 MBq/mL and one or more bismuth sequestering agent(s) in a total concentration of from about 0.03 mg/mL to about 0.7 mg/mL. Even more preferably, in certain embodiments, the pharmaceutical composition of the present disclosure comprises ²²⁵Ac in volumetric activity of from about 1 MBq/mL and one or more bismuth sequestering agent(s) in a total concentration of from about 0.05 mg/mL. The preferred sequestering agent herein is DMSA. In certain embodiments in connection with the disclosure herein, the sequestering agent is not DTPA. “About” herein means±20%, preferably ±10%, more preferably ±10% with regard to the volumetric activity and ±5% with regard to the bismuth chelating agent. The amounts of the sequestering agent referred to herein, may be the sequestering agents are disclosed herein as free acids or in their salt form, e.g. in the sodium (Na) salt, preferably, the amounts refer to the free acids.

In a particular embodiment, the pharmaceutical composition further comprises at least one stabilizer against radiolytic degradation, for example one or two stabilizers against radiolytic degradation.

Particularly, said one or more stabilizer against radiolytic degradation (antioxidant) can be selected from the group consisting of gentisic acid (2,5-dihydroxybenzoic acid) or salts thereof, ascorbic acid (L-ascorbic acid, vitamin C) or salts thereof (e.g. sodium ascorbate), methionine, histidine, melatonine, ethanol, and Se-methionine, and mixtures thereof, particularly selected from gentisic acid or salts thereof, and ascorbic acid or salts thereof. Preferably, only ascorbic acid or sodium ascorbate is being used as stabilizer/antioxidant. Preferably, ethanol is not being used as stabilizer. Preferably, in certain embodiments, ethanol is not being component of the pharmaceutical compositions.

Particularly, said at least two stabilizers can be gentisic acid or salts thereof and ascorbic acid or salts thereof.

In a particular embodiment of the pharmaceutical composition, the ratio between gentisic acid or salts and ascorbic acid or salts can be between 1:150 and 1:1, particularly between 1:50 and 1:2, more particularly between 1:4 and 2:5.

Particularly, said gentisic acid or salts thereof can be present at a concentration of least 300 μg/mL, particularly between 300 μg/mL and 5000 μg/mL, even more particularly about 1000 μg/mL.

Particularly, said ascorbic acid or salts thereof can be present at a concentration of at least 600 μg/mL, particularly between 600 μg/mL and 60000 μg/mL, even more particularly about 2000 μg/mL.

As a result, in a particular embodiment the pharmaceutical composition contains gentisic acid or salts thereof and ascorbic acid or salt thereof, said gentisic acid or salts thereof can be present at a concentration between 300 μg/mL and 5000 μg/mL, particularly about 1000 μg/mL and ascorbic acid or salts thereof can be present at a concentration between 600 μg/mL and 50000 μg/mL, particularly about 2000 μg/mL.

In a particular embodiment, the pharmaceutical composition can have a radiochemical purity (RCP) higher than 90% up to 96 hours, particularly higher than 92% up to 72 h, particularly higher than 95% for up to 72 h, preferably for up to 96 h, more preferably for up to 120 h or even 144 h.

In a particular embodiment, the pharmaceutical composition further comprises a buffer, particularly said buffer is an acetate buffer or a tris buffer, particularly in an amount to result in a concentration of from 0.3 to 0.7 mg/mL (particularly about 0.48 mg/mL) acetic acid and from 0.4 to 0.9 mg/mL (particularly about 0.66 mg/mL) sodium acetate. In certain embodiments, the pharmaceutical composition further comprises a TRIS buffer providing a pH of from about 7 to about 9, preferably, a pH of from about 7.5 to about 8.

The pharmaceutical composition according to the disclosure typically has a shelf life of at least 24 hours (h) at ≤25° C., at least 48 h at ≤25° C., at least 72 h at ≤25° C., of from 24 h to 120 h at ≤25° C., from 24 h to 96 h at ≤25° C., from 24 h to 84 h at ≤25° C., from 24 h to 72 h at ≤25° C., in particular has a shelf life of 96 h at ≤25° C. In certain embodiments, the pharmaceutical compositions have a radiochemical purity (RCP), as determined by iTLC (example for an analytical method is provided in the example section herein) or determined by HPLC, of ≥95% for at least 72 h (3 days), more preferably for at least 96 h (4 days), even more preferably for at least 120 h (5 days), even more preferably for at least 144 h (6 days), when stored at ≤25° C.

In a certain embodiment, the present disclosure provides a pharmaceutical composition (e.g. in the form of an aqueous solution) comprising of:
(a) a complex formed by
   (i) a ²²⁵Ac radionuclide, and,
   (ii) a target binding moiety linked to a chelating agent, preferably a PSMA binding moiety linked to a DOTA comprising chelating agent, preferably PSMA-R2, PSMA-617, or PSMA I&T;
   in a volumetric activity of about 0.5-2 MBq/mL, preferably about 1 MBq/mL (±10%);
(b) one or more sequestering agent(s), typically capable of sequestering Bi³⁺, preferably in a total concentration of 0.01-0.1 mg/mL, preferably 0.015-0.08 mg/mL, preferably about 0.05 mg/mL (±10%), preferably only DMSA is being used as sequestering agent, preferably DTPA is not being used in the pharmaceutical composition;
(c) optionally, one or more stabilizer(s)/antioxidant(s), preferably selected from the group consisting of gentisic acid and salts thereof, ascorbic acid and salts thereof, and mixtures thereof, preferably in a total concentration of 0.5 to 10 mg/mL, preferably 0.9 to 5 mg/mL, more preferably 1.3 to 3.8 mg/mL, preferably only ascorbic acid or a salt thereof is being used as stabilizer;
(d) optionally, a buffer for providing a pH of from 7 to 9, preferably a pH of from 7.5 to 8.5, more preferably a pH from 7.5 to 8.0, preferably said buffer is a TRIS buffer (e.g. composed of Tris hydrochloride, Tris, and/or Trometamol), preferably in a concentration of 0.5 to 5 mg/mL, or 0.05 to 0.5 M, more preferably, 0.1 to 0.25 M.
   wherein the pharmaceutical composition has a radiochemical purity (RCP), as determined by iTLC or determined by HPLC, of ≥95% for at least 72 h (3 days) when stored at ≤25° C., preferably, for at least 120 h (5 days).

Generally, the pharmaceutical composition according to the disclosure is produced at commercial scale manufacturing, in particular is produced at a batch size of at least 0.1 GBq, at least 5 GBq, at least 7 GBq.

In a particular embodiment, the pharmaceutical composition according to the disclosure is ready-to-use.

More specifically, the pharmaceutical composition according to the disclosure may be for commercial use.

According to an embodiment the pharmaceutical composition is an aqueous solution, for example an injectable formulation. According to a particular embodiment, the pharmaceutical composition is a solution for infusion.

The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and SHP Handbook on Injectable Drugs, Trissel, 15th ed., pages 622-630 (2009)).

The disclosure also relates to the pharmaceutical composition as described above for use in treating or preventing cancer, in particular SSTR2, PSMA, GRPR expressing cancers, in particular neuroendocrine tumors or prostate or breast cancer.

In another aspect of the disclosure, the pharmaceutical composition is produced at commercial scale manufacturing, in particular is produced at a batch size of at least 18.5 GBq (0.5 Ci), at least 37 GBq (1 Ci), or at least 55.5 GBq (1.5 Ci) and not more than 148 GBq (4 Ci), 129.5 GBq (3.5 Ci), 111 GBs (3 Ci), 92.5 GBq (2.5 Ci) or 74 GBq (2 Ci). Typically, it is produced at a batch size between 18.5 GBq (0.5 Ci) and 148 GBq (4 Ci).

In another aspect of the disclosure, the pharmaceutical composition is for commercial use.

In a further aspect, the disclosure also relates a pharmaceutical composition comprising a radiolabeled PSMA binding ligand linked to a chelating agent, typically [²²⁵Ac]Ac-PSMA binding ligand linked to a chelating agent, typically the PSMA-binding ligand of formula (II) or (III) for use in treating or preventing cancer in a subject in need thereof, wherein said pharmaceutical composition is formulated with stabilizers as described in any of the previous embodiments, and is administered to said subject at a therapeutically efficient amount comprised between 0.5 mCi and 1000 mCi, particularly between 50 mCi and 400 mCi, typically with a radiochemical purity (RCP) superior to 95% at the time of administration.

In certain aspects the subject is a mammal, for example but not limited to a rodent, canine, feline, or primate. In particular aspects, the subject is a human.

In specific embodiments, a therapeutically efficient amount of the composition is administered to said subject 1 to 8 times per treatment, particularly 3 times per treatment.

For example, a human patient may be treated with said pharmaceutical composition comprising a radiolabeled PSMA binding ligand linked to a chelating agent, specifically [²²⁵Ac]Ac-PSMA binding ligand linked to a chelating agent, typically the PSMA-binding ligand of formula (II) or (III), administered intravenously in 2 to 8 cycles of a 0.5 mCi to 1000 mCi each, typically with radiochemical purity (RCP) superior to 95% at the time of administration.

In certain instances, the pharmaceutical composition of the present disclosure can be used in combination with other therapeutic agents, such as other anti-cancer agents, anti-allergic agents, anti-nausea agents, anti-emetic agents, agents against metal toxicity, pain relievers, cytoprotective agents, and mixtures thereof.

In a particular embodiment the agents against metal toxicity, the bismuth sequestering agents, are selected from chelators such as, but not limited to (alternatively: are selected from the list of chelators consisting of), the dithiol chelators 2,3 dimercapto-1-propane sulfonic acid (DMPS), meso 2,3-dimercapto succinic acid (DMSA), ethylenediamine tetra-acetic acid (EDTA), diethylenetriamine pentaacetic acid (DTPA), or salts of any of those chelators, calcium diethylenetriamine pentaacetic acid (Ca-DTPA) and zinc diethylenetriamine pentaacetic acid (Zn-DTPA) or diuretics such as, but not limited to, furosemide, chlorthiazide, hydrochlorothiazide and bumex. Preferably, DMSA is used. In certain embodiments, DTPA is not used.

In a particular embodiment the agents against metal toxicity may be administered prior, co-concomitantly or after the administration of the pharmaceutical composition according to the disclosure, particularly after the administration of the pharmaceutical composition according to the disclosure.

### A Method for Preparing a Pharmaceutical Composition According to the Disclosure

As previously mentioned, the disclosure also concerns a method for preparing said pharmaceutical composition, comprising the steps of
1) Forming a complex of the radionuclide and the chelating agent linked to the target binding organic moiety by
(1.1) providing an aqueous solution comprising the radionuclide;
(1.2) providing an aqueous solution comprising the chelating agent linked to the target binding organic moiety; and
(1.3) mixing the solutions obtained in steps (1.1) and (1.2) and heating the resulting mixture;
2) Diluting the complex solution obtained at the end of step (1) by mixing the complex solution obtained at the end of step (1) with a dilution solution,
   wherein said bismuth sequestering agent is comprised in the aqueous solution of step 1.1 and/or the aqueous solution of step 1.2 and/or the dilution solution of step 2.

In a particular embodiment, the bismuth sequestering agent is comprised in the aqueous solution of step 1.2 and/or the dilution solution of step 2.

In a particular embodiment, the bismuth sequestering agent is comprised in the dilution solution of step 2.

In a particular embodiment, the solution of step (1.2) further comprises a buffer, particularly an acetate buffer or a tris buffer, preferably a TRIS buffer, preferably a TRIS buffer providing a pH of from about 7 to about 9, preferably a pH from about 7.5 to about 8.5, more preferably a pH of about 8.

Particularly, in step (1.3) the resulting mixture is heated to a temperature of 70 to 99° C., particularly from 90 to 98° C., for 2 to 59 min, preferably from 90 to 98° C. for 10 to 30 min, more preferably about 95° C. for about 20 min.

In a particular embodiment, the solution of step (1.1) comprises AcCl₃, preferably ²²⁵AcCl₃, more preferably ²²⁵AcCl₃ in 0.1 N HCl.

Particularly, the solution of step (1.2) comprises ²²⁵Ac radiolabeled PSMA binding complex and gentisic acid. Particularly, the solution of step (1.2) comprises ²²⁵Ac radiolabeled PSMA binding complex and optionally gentisic acid.

Particularly, the dilution solution of step (2) comprises said bismuth sequestering agent, and ascorbic acid and saline. Particularly, the dilution solution of step (2) comprises said bismuth sequestering agent, and optional ascorbic acid and optionally saline.

In a particular embodiment, the process according to the disclosure, further comprising the following steps:
(3) Filtering the solution obtained by step (2) through 0.2 μm; and,
(4) Dispensing the filtered solution obtained by step (3) into dose unit containers in a volume required to deliver the radioactive dose of from 4.0 to 15 MBq, particularly from 7.0 to 8.0 MBq, more particularly from 7.3 to 7.7 MBq, even more particularly from 7.4-7.5 MBq, particularly said volume can be from 5 to 50 mL, more particularly from 6 to 30 mL, even more particularly from 7 to 16 mL.

Particularly, the dose unit containers in step (4) are stoppered vials, enclosed within a lead container.

### EXAMPLES

### Example 1: Formulation with [225Ac]Ac-PSMA-R2 and DTPA

PSMA-R2 compound is synthesized as described in WO2017/165473.

The formulation comprising [²²⁵Ac]Ac-PSMA-R2 with DTPA is prepared according to following Table. It is a ready to use 1 MBq/ml solution for injection/infusion.

| Component | Purpose | Quantity | |
|---|---|---|---|
| PSMA-R2 | Precursor of active | 8 | μg/mL |
| | substance | | |
| [²²⁵Ac]Ac-PSMA-R2 | Drug substance | 2.46 ng/mL at EOS* | |
| DTPA | Sequestering Agent | 0.03-0.04 | mg/mL |
| Ascorbic acid | Radiolysis | 1.73-2.38 | mg/mL |
| | protector/anti-oxidant | | |
| Sodium hydroxide | pH adjuster | 0.40-0.54 | mg/mL |
| Tris hydrochloride | Buffering Agent | 2.33-0.79 | mg/mL |
| Trometamol | Buffering Agent | 1.48-0.50 | mg/mL |
| Water For Injection | Solvent | q.s.** | |
| Sodium chloride | Isotonizing agent | 6.98-7.72 | mg/mL |

(NaCl) 0.9%

*EOS is the End of Synthesis. Values calculated assuming 225Ac specific activity of 487.7 MBq/nmol at labeling time and a theoretical synthesis yield of 100% and radiochemical purity ≥ 97%
**This amount includes as well the water for injections filled in the bulk solution bottle (0.5 ± 0.1 mL) and in the primary packaging (0.20 ± 0.02 mL) during the sterilization process as added amount is considered negligible.

### Example 2: Formulation with [²²⁵Ac]Ac-PSMA-R2 and DMSA

### Radiolabeling Scheme:

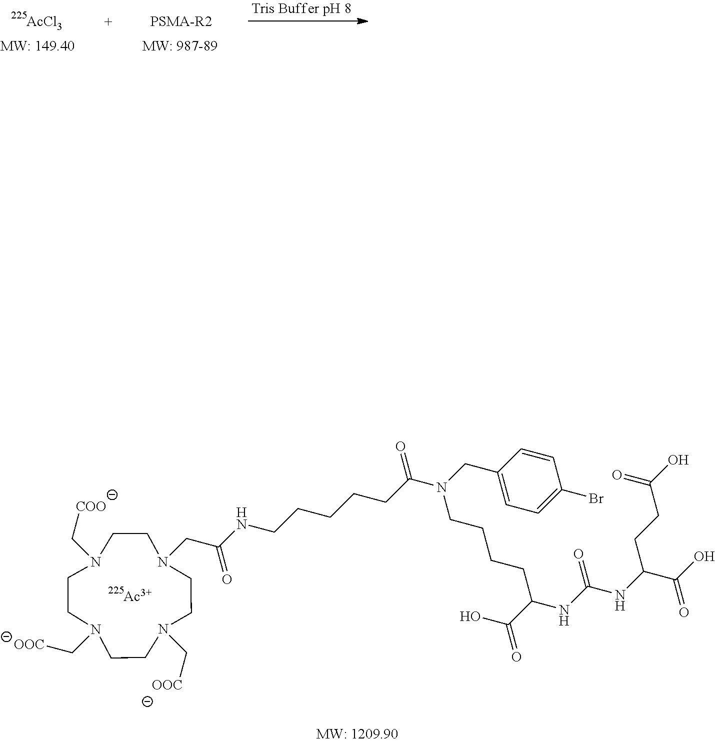

### PSMA-R2 Reconstitution

To a vial containing 1 mg PSMA-R2 was added 1 mL of water to obtain a solution 1000 ppm.

### Drug Substance (DS) Preparation

Labelling: In a 10 mL glass vial 120 μL of ²²⁵AcCl₃ 0.1 N HCl (calibration: day 1, 08:00, 91.49 MBq, 1186 μL) was added. Glass vial was crimped and measured at dose calibrator (5.925 MBq, day 7, 14:48). Solution of PSMA-R2 (47 μL) was added then followed by TRIS buffer 0.25 M pH 8 (318 μL). pH of resulting reaction mixture (tot. volume 485 μL) was measured via pH strip (Macherey-Nagel pH-Fix 7.5-9.5): pH 7.9. Reaction mixture was then heated at 95° C. using heating block (Labnet, AccuBlockDigital Dry Bath) for 20 min. Solution was allowed to cool down to ambient temperature over 10 min. Radiolabeling was performed with a ratio mass peptide in mircogramm to activity in MBq of 8; mass peptide 47.4 microgram; MBq/microgram peptide at time of use 0.125; MBq/microgram peptide (ART) 1.270.

### Preparation of DMSA Solution

1.79 mg of DMSA (meso-2,3-dimercaptosuccininc acid) (Sigma Aldrich) was dispensed in a 1.5 mL centrifuge tube. 0.895 mL of water was added. Suspension was agitated using vortex mixer until complete dissolution to obtain a homogeneous solution of DMSA at 2 mg/mL.

### Preparation of Sodium Ascorbate Solution

L-ascorbic acid (302.34 mg, 1.7 mmol) and sodium hydroxide (69.85 mg, 1.7 mmol) were dispensed on a pre-tared balance, then transferred to a 50 mL falcon vial. Water (11.72 mL) was added to obtain a solution of Sodium Ascorbate at 26.83 mg/mL.

### Drug Product (DP) Formulation

Sodium ascorbate (596 μL) and DMSA (148 μL) solutions were dispensed via pipette and transferred into a 1.5 mL centrifuge tube. The obtained solution was transferred to a 10 mL reaction vial (containing [²²⁵Ac]Ac-PSMAR2 DS solution) through 1 mL syringe. The syringe was used to transfer 3×1 mL of saline (sodium chloride 0.9%) into reaction vial. Finally, 1.695 mL of saline was added to obtain a solution of 1 MBq/mL. The final volume of the formulation was 5.925 mL. The final pH of the formulation was 7.5.

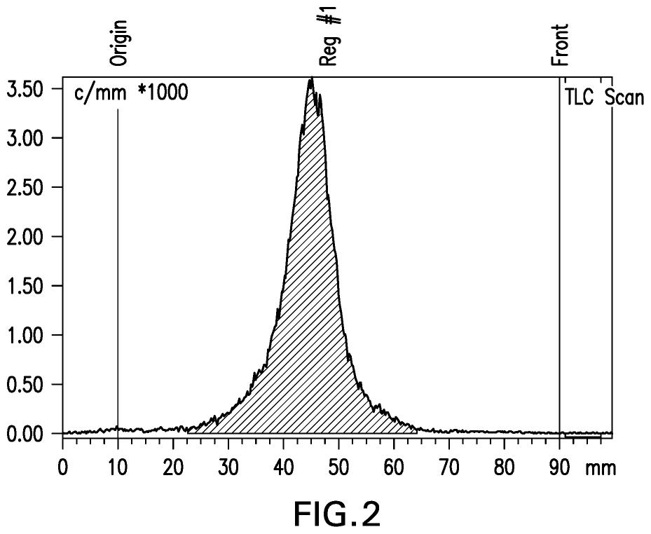

A small aliquot was dispensed (˜100 μL) for iTLC analysis (RP-18 F254S, NH4OAc 5M Aq/H2O/MeOH 3:2:7.5, described in more details below). The acquisition of TLC plate by alpha scanner was carried out at >18 h post development. This time is provided for 225Ac to reach secular equilibrium, and for migrated daughters to decay. The radio-iTLC analysis indicated >99% radiochemical purity (RCP), see FIG. 2.

### Stability Study

With the aim of having stability data of [²²⁵Ac]Ac-PSMA-R2 DP with DMSA comparable with DTPA formulation, it has been chosen to [²²⁵Ac]Ac-PSMA-R2 DP with DMSA undergo under the same conditions of stability with DTPA formulation, such as 5 mL solution volume, stored at 25° C. Thus, a solution of drug product was removed via syringe (˜900 μL) in order to obtain a volume of 5 mL in the drug product vial. Vial was then placed into lead container and stored in a 25° C. chamber. Radiochemical purity was analysed by iTLC (RP-18 F254S, NH40Ac 5M Aq/H2O/MeOH 3:2:7.5, plate length: 100 mm, sample run: 80 mm (from 10 to 90 mm), sample volume: 90 microliter, activity deposit: 1.2-2.0 KBq, scanning time post-development >18 h, TLC scanner: MiniGita 37292, detector alpha: PMT+Plastic+ZnS, Rev 1.11, SN 17115, acquisition time 10 min) over 7 days (time points: 24 h, 48h, 72h, 144h, 168 h).

### Results:

The following table provides the stability test data by iTLC analysis for the DMSA formulation.

| | Volume | | | |
|---|---|---|---|---|
| Days after | deposit | Activity Deposit (KBq) | | |
| synthesis | (μL) | (at time of development) | Rf | RCP(%) |
| t0 | 2 | 2.00 | 0.438 | 100.00% |
| Day 1 (24 h) | 2 | 1.87 | 0.441 | 98.59% |
| Day 2 (48 h) | 2 | 1.74 | 0.444 | 98.34% |
| Day 3 (72 h) | 2 | 1.62 | 0.438 | 98.01% |
| Day 4 (96 h) | N/A | N/A | N/A | N/A |
| Day 5 (120 h) | N/A | N/A | N/A | N/A |
| Day 6 (144 h) | 2 | 1.32 | 0.431 | 96.40% |
| Day 7 (168 h) | 2 | 1.23 | 0.466 | 93.20% |

The following table provides comparable RCP data of DMSA formulation with the DTPA formulation, under same conditions (5 mL solution at 25° C.). The data indicate that both, DTPA and DMSA, provide good RCP over time. Compared to DTPA, DMSA however seems to provide good RCP over a longer time period.

| Days after | RCP(%) | RCP(%) |
|---|---|---|
| synthesis | DMSA formulation (5 mL) | DTPA formulation (5 mL) |
| t0 | 100.00% | 100.00% |
| Day 1 (24 h) | 98.59% | 99.65% |
| Day 2 (48 h) | 98.34% | N/A |
| Day 3 (72 h) | 98.01% | N/A |
| Day 4 (96 h) | N/A | 97.91% |
| Day 5 (120 h) | N/A | 93.04% |
| Day 6 (144 h) | 96.40% | N/A |
| Day 7 (168 h) | 93.20% | N/A |

1. A pharmaceutical composition comprising
(a) a ²²⁵Ac radiolabelled complex formed by
(i) an ²²⁵Ac radionuclide, and,
(ii) a target binding moiety linked to a chelating agent; and
(b) a bismuth sequestering agent, typically capable of sequestering Bi³⁺
(c) optionally an antioxidant, particularly chosen in the group comprising gentisic acid and salts thereof, ascorbic acid and salts thereof, and mixtures thereof.

2. The pharmaceutical composition of claim 1, wherein said antioxidant is chosen in the group comprising gentisic acid and salts thereof, ascorbic acid and salts thereof, and mixtures thereof.

3. The pharmaceutical composition according to claim 1, wherein said sequestering agent is a chelating agent for Bi³⁺ having binding kinetics for Bi³⁺ which is higher than the corresponding binding kinetics of DTPA and/or DOTA.

4. The pharmaceutical composition according to claim 1, wherein said sequestering agent is a chelating agent having binding selectivity of Bi³⁺ over ²²⁵Ac³⁺.

5. The pharmaceutical composition according to claim 1, wherein the bismuth sequestering agent is a chelating agent selected from the group consisting of DMSA, DOTA, DTPA, CHX-A″″-DTPA, L^{py}, L^{pyd}, L^{pyr}, L^{pz}, NETA, 3p-C-NETA, DEPA, 3p-C-DEPA, and C-DEPA.

6. The pharmaceutical composition according to claim 1, wherein said radionuclide is present at a concentration so that it provides a volumetric radioactivity of at least 5 MBq/mL.

7. The pharmaceutical composition according to claim 1, wherein the molar ratio of (i) the ²²⁵Ac radiolabelled complex to (ii) the bismuth sequestering agent is comprised between 1:8500 and 1:80000.

8. The pharmaceutical composition according to claim 1, further comprising at least one stabilizer against radiolytic degradation.

9. The pharmaceutical composition according to claim 8, wherein said one or more stabilizer against radiolytic degradation is or are selected from the group consisting of gentisic acid (2,5-dihydroxybenzoic acid) or salts thereof, ascorbic acid (L-ascorbic acid, vitamin C) or salts thereof (e.g. sodium ascorbate), methionine, histidine, melatonine, ethanol, and Se-methionine, and mixtures thereof.

10. The pharmaceutical composition according to claim 1, comprising at least two stabilizers against radiolytic degradation, wherein said at least two stabilizers are gentisic acid or salts thereof and ascorbic acid or salts thereof.

11. The pharmaceutical composition according to claim 10, wherein the ratio between gentisic acid or salts and ascorbic acid or salts is between 1:32 and 1:1.

12. The pharmaceutical composition according to claim 11, wherein said gentisic acid or salts thereof is present at a concentration of least 300 μg/mL.

13. The pharmaceutical composition according to claim 12, wherein said ascorbic acid or salts thereof is present at a concentration of at least 600 μg/mL.

14. The pharmaceutical composition according to claim 1, wherein the chelating agent of the ²²⁵Ac radiolabeled complex is selected from DOTA, DTPA, NTA, EDTA, DO3A, and NOTA.

15. The pharmaceutical composition according to claim 1, wherein said target binding moiety is selected among PSMA binding ligands, somatostatin receptor binding peptides, gastrin-releasing peptide receptor antagonists and integrin.

16. The pharmaceutical composition according to claim 1, wherein said pharmaceutical composition has a radiochemical purity higher than 90% up to 96 hours.

17. The pharmaceutical composition according to claim 1, further comprising a buffer, wherein said buffer is an acetate buffer or a tris buffer.

18. A patient dose unit comprising
(a) 5-20 MBq of ²²⁵Ac;
(b) 0.15-0.80 mg of bismuth sequestering agent; and
(c) optionally 9-50 mg of antioxidant(s).

19. A process for preparing a pharmaceutical composition according to claim 1, comprising the steps of
1) Forming a complex of the radionuclide and the chelating agent linked target binding organic moiety by
(1.1) providing an aqueous solution comprising the radionuclide;
(1.2) providing an aqueous solution comprising the chelating agent linked target binding organic moiety; and
(1.3) mixing the solutions obtained in steps (1.1) and (1.2) and heating the resulting mixture;
2) Diluting the complex solution obtained by step (1) by mixing the complex solution obtained by step (1) with a dilution solution,
wherein said bismuth sequestering agent is comprised in the aqueous solution of step 1.1 and/or the aqueous solution of step 1.2 and/or the dilution solution of step 2.

20. The process according to claim 19, wherein said bismuth sequestering agent is comprised in the dilution solution of step 2.

21. The process according to claim 20, further comprising the process steps:
(3) Filtering the solution obtained by step (2) through 0.2 μm; and,
(4) Dispensing the filtered solution obtained by step (3) into dose unit containers in a volume required to deliver the radioactive dose of from 4.0 to 15 MBq.
